(19) United States
(12) Patent Application Publication
(10) Pub. No.: US 2021/0118572 A1
(43) Pub. Date: Apr. 22, 2021

(54) System and Method for Mapping Cardiac Activity

(71) Applicant: St. Jude Medical, Cardiology Division, Inc., St. Paul, MN (US)

(72) Inventors: Valtino X. Afonso, Oakdale, MN (US); Frank Miller, Roseville, MN (US); William Choe, Highlands Ranch, CO (US); Charles Boorman, Denver, CO (US); Austin Davies, Englewood, CO (US); Nate Mullins, Highlands Ranch, CO (US); Sri Sundaram, Englewood, CO (US); Austin Stucky, Denver, CO (US)

(21) Appl. No.: 17/054,638

(22) PCT Filed: Jun. 7, 2019
(86) PCT No.: PCT/US2019/036158

§ 371 Date: Nov. 11, 2020

Related U.S. Application Data
(60) Provisional application No. 62/685,164, filed on Jun. 14, 2018.

(51) Int. Cl. G16H 50/50; A61B 5/00; G06T 19/20

(52) U.S. Cl. CPC G16H 50/50; A61B 5/742; G06T 2219/2021; G06T 19/20; G06T 2210/41; A61B 5/7475

(57) ABSTRACT

Two or more electrophysiology characteristics can be graphically represented in a single representation output, for example, by an electroanatomical mapping system. The system can generate or receive multiple electrophysiology maps, one for each of a corresponding number of electrophysiological characteristics. The system can also generate or receive a three-dimensional anatomical model, such as a cardiac surface model, that includes a focal point. The system can identify a display region about the focal point and transform the display region from a three-dimensional surface into a plane. One or more of the electrophysiology maps can be represented by varying the elevation of the plane, e.g., according to value(s) of the represented electrophysiological characteristic(s). One or more additional electrophysiology maps can be represented on the elevation-varied plane, e.g., in color scale, grey scale, or the like.

### CROSS-REFERENCE TO RELATED APPLICATIONS

This application claims the benefit of U.S. provisional application No. 62/685,164, filed 14 Jun. 2018, which is hereby incorporated by reference as though fully set forth herein.

### BACKGROUND

The present disclosure relates generally to electrophysiological mapping, such as may be performed in cardiac diagnostic and therapeutic procedures. In particular, the present disclosure relates to systems, apparatuses, and methods for graphically representing multiple electrophysiological characteristics (e.g., local activation time, peak-to-peak voltage, or the like) on a single cardiac geometry.

Electrophysiological mapping, and more particularly electrocardiographic mapping, is a part of numerous cardiac diagnostic and therapeutic procedures. As the complexity of such procedures increases, however, the electrophysiology maps utilized must increase in quality, in density, and in the rapidity and ease with which they can be generated.

Extant electroanatomical mapping systems can often provide geometry models representing the cardiac anatomy. Extant electroanatomical mapping systems also are often capable of rendering these geometries in color scale, grey scale, stippling, or the like in order to represent an electrophysiological characteristic, such as local activation time, peak-to-peak voltage, or the like.

It can be desirable to evaluate multiple electrophysiological characteristics as part of an electrophysiology study. It can be difficult or visually overwhelming, however, to render a graphical representation of multiple electrophysiological characteristics on a single surface geometry.

### BRIEF SUMMARY

Disclosed herein is a method of graphically representing multiple electrophysiological characteristics on a single surface model. The method includes the steps of: receiving a three-dimensional model of a cardiac surface; identifying a focal point within the three-dimensional model of the cardiac surface; identifying a display region of the three-dimensional model of the cardiac surface around the focal point; transforming the display region from a three-dimensional model into a plane; and graphically representing a first electrophysiological characteristic by varying an elevation of the plane according to values of the first electrophysiological characteristic. The method can also include graphically representing a second electrophysiological characteristic on the elevation-varied plane.

According to aspects of the disclosure, the step of identifying a display region of the three-dimensional model of the cardiac surface around the focal point includes: propagating a geodesic wavefront through the three-dimensional model of the cardiac surface, originating at the focal point; and adding polygons of the three-dimensional model of the cardiac surface through which the geodesic wavefront passes to the display region. The step of propagating the geodesic wavefront through the three-dimensional model of the cardiac surface can end after the geodesic wavefront propagates a preset geodesic distance from the focal point (e.g., between about 4 cm and about 6 cm, such as about 5 cm). Alternatively, the step of propagating the geodesic wavefront through the three-dimensional model of the cardiac surface can end when the geodesic wavefront intersects itself.

In embodiments of the disclosure, the step of transforming the display region from a three-dimensional model into a plane includes computing a continuous one-to-one mapping from the three-dimensional model of the cardiac surface to a plane using a transformation algorithm. Suitable transformation algorithms include, without limitation, least squares conformal mapping algorithms and local/global approach to mesh parameterization algorithms.

The step of identifying a focal point within the three-dimensional model of the cardiac surface can include accepting user input designating the focal point within the three-dimensional model of the cardiac surface. Alternatively, the step of identifying a focal point within the three-dimensional model of the cardiac surface can include identifying the focal point within the three-dimensional model of the cardiac surface according to a viewing orientation of the three-dimensional model of the cardiac surface.

It is contemplated that the plane can be tangent to the cardiac surface at the focal point. Further, it is contemplated that the step of varying an elevation of the plane according to values of the first electrophysiological characteristic can include displacing points in the plane in a direction normal to the plane according to values of the first electrophysiological characteristic.

Also disclosed herein is a method of graphically representing two electrophysiology maps in a single representation, including the steps of: receiving a first electrophysiology map of a first electrophysiological characteristic; receiving a three-dimensional cardiac surface model; identifying a focal point in the three-dimensional cardiac surface model; transforming a display region about the focal point from a three-dimensional cardiac surface into a plane; and graphically representing the first electrophysiology map of the first electrophysiological characteristic by varying an elevation of the plane. The method can also include: receiving a second electrophysiology map of a second electrophysiological characteristic; and graphically representing the second electrophysiology map of the second electrophysiological characteristic on the elevation-varied plane.

According to aspects disclosed herein, the step of transforming a display region about the focal point from a three-dimensional cardiac surface into a plane can include: identifying the display region by propagating a geodesic wavefront from the focal point; and computing a continuous one-to-one mapping from the three-dimensional model of the cardiac surface to a plane using a transformation algorithm. In turn, the step of propagating a geodesic wavefront from the focal point can include propagating the geodesic wavefront from the focal point to a preset geodesic distance from the focal point. Alternatively, the step of propagating a geodesic wavefront from the focal point can include propagating the geodesic wavefront from the focal point until the geodesic wavefront intersects itself. In general, and according to embodiments disclosed herein, the step of identifying the display region by propagating a geodesic wavefront from the focal point can include adding polygons of the three-dimensional model of the cardiac surface passed by the propagating geodesic wavefront to the display region.

The instant disclosure also provides a system for graphically representing multiple electrophysiological characteristics on a single surface model. The system includes a modeling module configured to: receive a three-dimensional model of a cardiac surface, the three-dimensional model including a focal point; identify a display region of the three-dimensional model of the cardiac surface around the focal point; transform the display region from the three-dimensional model into a plane; and output a graphical representation of a first electrophysiological characteristic by varying an elevation of the plane according to values of the first electrophysiological characteristic. The modeling module can also be configured to output a graphical representation of a second electrophysiological characteristic on the elevation-varied plane.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a schematic diagram of an exemplary electroanatomical mapping system.

FIG. 2 depicts an exemplary catheter that can be used in connection with aspects of the instant disclosure.

FIG. 3 is a flowchart of representative steps that can be followed according to exemplary embodiments disclosed herein.

FIG. 4 illustrates a three-dimensional surface model of a cardiac region and a focal point thereon.

FIG. 5 illustrates a display region about the focal point of FIG. 4, in accordance with aspects of the teachings herein.

FIG. 6 illustrates a transformation of the display region of FIG. 5 into a plane, in accordance with aspects of the teachings herein.

FIGS. 7A through 7D are exemplary graphical representations of a local activation time map (FIG. 7A), a peak-to-peak voltage map (FIG. 7B), a local activation time map expressed on a planar surface (FIG. 7C), and a concurrent map of local activation time and peak-to-peak voltage, according to aspects of the teachings herein (FIG. 7D).

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

The instant disclosure provides systems, apparatuses, and methods for the creation of electrophysiology maps (e.g., electrocardiographic maps) that provide information regarding cardiac activity. In particular, the instant disclosure provides systems, apparatuses, and methods for displaying multiple electrophysiological characteristics on a single surface model. Although certain local activation time and peak-to-peak voltage will be used herein as illustrative electrophysiological characteristics, the teachings herein can be applied to the graphical representation of substantially any scalar quantity on a geometric model, including, without limitation, catheter pressure information and complex fractionated atrial electrogram (CFAE) information.

For purposes of illustration, aspects of the disclosure will be described in detail herein in the context of a cardiac mapping procedure carried out using an electrophysiology mapping system (e.g., using an electroanatomical mapping system such as the EnSite Precision™ cardiac mapping system from Abbott Laboratories of Abbott Park, Ill.).

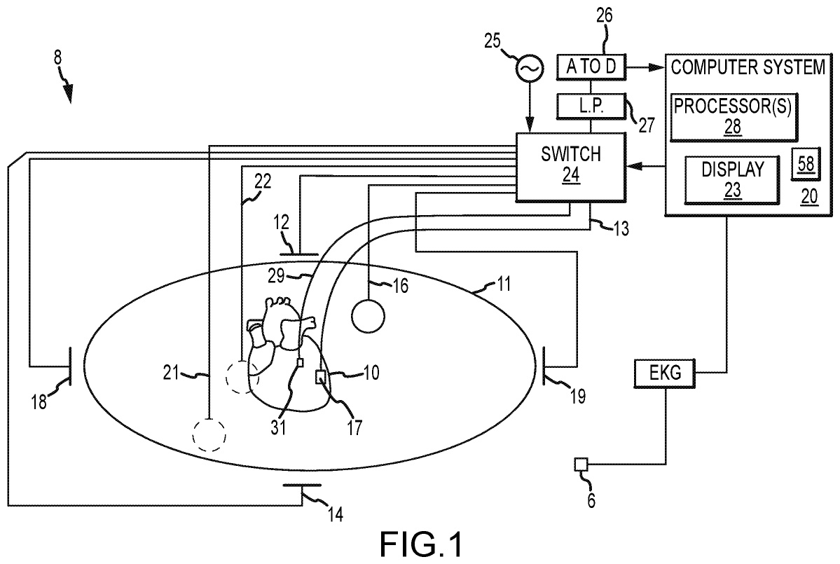

FIG. 1 shows a schematic diagram of an exemplary electroanatomical mapping system 8 for conducting cardiac electrophysiology studies by navigating a cardiac catheter and measuring electrical activity occurring in a heart 10 of a patient 11 and three-dimensionally mapping the electrical activity and/or information related to or representative of the electrical activity so measured. System 8 can be used, for example, to create an anatomical model of the patient's heart 10 using one or more electrodes. System 8 can also be used to measure electrophysiology data at a plurality of points along a cardiac surface and store the measured data in association with location information for each measurement point at which the electrophysiology data was measured, for example, to create a diagnostic data map of the patient's heart 10.

As one of ordinary skill in the art will recognize, and as will be further described below, system 8 determines the location, and in some aspects the orientation, of objects, typically within a three-dimensional space, and expresses those locations as position information determined relative to at least one reference.

For simplicity of illustration, the patient 11 is depicted schematically as an oval. In the embodiment shown in FIG. 1, three sets of surface electrodes (e.g., patch electrodes) are shown applied to a surface of the patient 11, defining three generally orthogonal axes, referred to herein as an x-axis, a y-axis, and a z-axis. In other embodiments the electrodes could be positioned in other arrangements, for example multiple electrodes on a particular body surface. As a further alternative, the electrodes do not need to be on the body surface, but could be positioned internally to the body.

In FIG. 1, the x-axis surface electrodes 12, 14 are applied to the patient along a first axis, such as on the lateral sides of the thorax region of the patient (e.g., applied to the patient's skin underneath each arm) and may be referred to as the Left and Right electrodes. The y-axis electrodes 18, 19 are applied to the patient along a second axis generally orthogonal to the x-axis, such as along the inner thigh and neck regions of the patient, and may be referred to as the Left Leg and Neck electrodes. The z-axis electrodes 16, 22 are applied along a third axis generally orthogonal to both the x-axis and the y-axis, such as along the sternum and spine of the patient in the thorax region, and may be referred to as the Chest and Back electrodes. The heart 10 lies between these pairs of surface electrodes 12/14, 18/19, and 16/22.

An additional surface reference electrode (e.g., a “belly patch”) 21 provides a reference and/or ground electrode for the system 8. The belly patch electrode 21 may be an alternative to a fixed intra-cardiac electrode 31, described in further detail below. It should also be appreciated that, in addition, the patient 11 may have most or all of the conventional electrocardiogram (“ECG” or “EKG”) system leads in place. In certain embodiments, for example, a standard set of 12 ECG leads may be utilized for sensing electrocardiograms on the patient's heart 10. This ECG information is available to the system 8 (e.g., it can be provided as input to computer system 20). Insofar as ECG leads are well understood, and for the sake of clarity in the figures, only a single lead 6 and its connection to computer 20 is illustrated in FIG. 1.

A representative catheter 13 having at least one electrode 17 is also shown. This representative catheter electrode 17 is referred to as the “roving electrode,” “moving electrode,” or “measurement electrode” throughout the specification. Typically, multiple electrodes 17 on catheter 13, or on multiple such catheters, will be used. In one embodiment, for example, the system 8 may comprise sixty-four electrodes on twelve catheters disposed within the heart and/or vasculature of the patient. In other embodiments, system 8 may utilize a single catheter that includes multiple (e.g., eight) splines, each of which in turn includes multiple (e.g., eight) electrodes.

The foregoing embodiments are merely exemplary, however, and any number of electrodes and/or catheters may be used. For example, in some embodiments, a high density mapping catheter, such as the Ensite™ Array™ non-contact mapping catheter of Abbott Laboratories, can be utilized.

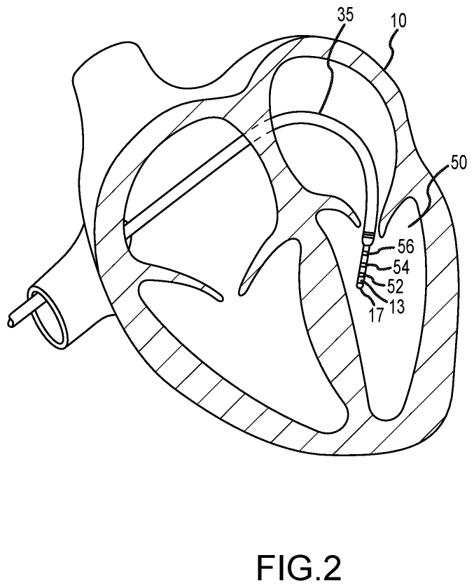

Likewise, it should be understood that catheter 13 (or multiple such catheters) are typically introduced into the heart and/or vasculature of the patient via one or more introducers and using familiar procedures. For purposes of this disclosure, a segment of an exemplary catheter 13 is shown in FIG. 2. In FIG. 2, catheter 13 extends into the left ventricle 50 of the patient's heart 10 through a transseptal sheath 35. The use of a transseptal approach to the left ventricle (e.g., across the intra-atrial septum and through the mitral valve) is well known and will be familiar to those of ordinary skill in the art, and need not be further described herein. Of course, catheter 13 can also be introduced into the heart in any other suitable manner, and may also be introduced into any chamber of the heart consistent with application of the teachings herein.

Catheter 13 includes electrode 17 on its distal tip, as well as a plurality of additional measurement electrodes 52, 54, 56 spaced along its length in the illustrated embodiment. Typically, the spacing between adjacent electrodes will be known, though it should be understood that the electrodes may not be evenly spaced along catheter 13 or of equal size to each other. Since each of these electrodes 17, 52, 54, 56 lies within the patient, location data may be collected simultaneously for each of the electrodes by system 8.

Similarly, each of electrodes 17, 52, 54, and 56 can be used to gather electrophysiological data from the cardiac surface (e.g., surface electrograms). The ordinarily skilled artisan will be familiar with various modalities for the acquisition and processing of electrophysiology data points (including, for example, both contact and non-contact electrophysiological mapping), such that further discussion thereof is not necessary to the understanding of the techniques disclosed herein. Likewise, various techniques familiar in the art can be used to generate a graphical representation of a cardiac geometry and/or of cardiac electrical activity from the plurality of electrophysiology data points. Moreover, insofar as the ordinarily skilled artisan will appreciate how to create electrophysiology maps from electrophysiology data points, the aspects thereof will only be described herein to the extent necessary to understand the present disclosure.

Returning now to FIG. 1, in some embodiments, an optional fixed reference electrode 31 (e.g., attached to a wall of the heart 10) is shown on a second catheter 29. For calibration purposes, this electrode 31 may be stationary (e.g., attached to or near the wall of the heart) or disposed in a fixed spatial relationship with the roving electrodes (e.g., electrodes 17), and thus may be referred to as a “navigational reference” or “local reference.” The fixed reference electrode 31 may be used in addition or alternatively to the surface reference electrode 21 described above. In many instances, a coronary sinus electrode or other fixed electrode in the heart 10 can be used as a reference for measuring voltages and displacements; that is, as described below, fixed reference electrode 31 may define the origin of a coordinate system.

Each surface electrode is coupled to a multiplex switch 24, and the pairs of surface electrodes are selected by software running on a computer 20, which couples the surface electrodes to a signal generator 25. Alternately, switch 24 may be eliminated and multiple (e.g., three) instances of signal generator 25 may be provided, one for each measurement axis (that is, each surface electrode pairing).

The computer 20 may comprise, for example, a conventional general-purpose computer, a special-purpose computer, a distributed computer, or any other type of computer. The computer 20 may comprise one or more processors 28, such as a single central processing unit (“CPU”), or a plurality of processing units, commonly referred to as a parallel processing environment, which may execute instructions to practice the various aspects described herein.

Generally, three nominally orthogonal electric fields are generated by a series of driven and sensed electric dipoles (e.g., surface electrode pairs 12/14, 18/19, and 16/22) in order to realize catheter navigation in a biological conductor. Alternatively, these orthogonal fields can be decomposed and any pairs of surface electrodes can be driven as dipoles to provide effective electrode triangulation. Likewise, the electrodes 12, 14, 18, 19, 16, and 22 (or any number of electrodes) could be positioned in any other effective arrangement for driving a current to or sensing a current from an electrode in the heart. For example, multiple electrodes could be placed on the back, sides, and/or belly of patient 11. Additionally, such non-orthogonal methodologies add to the flexibility of the system. For any desired axis, the potentials measured across the roving electrodes resulting from a predetermined set of drive (source-sink) configurations may be combined algebraically to yield the same effective potential as would be obtained by simply driving a uniform current along the orthogonal axes.

Thus, any two of the surface electrodes 12, 14, 16, 18, 19, 22 may be selected as a dipole source and drain with respect to a ground reference, such as belly patch 21, while the unexcited electrodes measure voltage with respect to the ground reference. The roving electrodes 17 placed in the heart 10 are exposed to the field from a current pulse and are measured with respect to ground, such as belly patch 21. In practice the catheters within the heart 10 may contain more or fewer electrodes than the sixteen shown, and each electrode potential may be measured. As previously noted, at least one electrode may be fixed to the interior surface of the heart to form a fixed reference electrode 31, which is also measured with respect to ground, such as belly patch 21, and which may be defined as the origin of the coordinate system relative to which system 8 measures positions. Data sets from each of the surface electrodes, the internal electrodes, and the virtual electrodes may all be used to determine the location of the roving electrodes 17 within heart 10.

The measured voltages may be used by system 8 to determine the location in three-dimensional space of the electrodes inside the heart, such as roving electrodes 17 relative to a reference location, such as reference electrode 31. That is, the voltages measured at reference electrode 31 may be used to define the origin of a coordinate system, while the voltages measured at roving electrodes 17 may be used to express the location of roving electrodes 17 relative to the origin. In some embodiments, the coordinate system is a three-dimensional (x, y, z) Cartesian coordinate system, although other coordinate systems, such as polar, spherical, and cylindrical coordinate systems, are contemplated.

As should be clear from the foregoing discussion, the data used to determine the location of the electrode(s) within the heart is measured while the surface electrode pairs impress an electric field on the heart. The electrode data may also be used to create a respiration compensation value used to improve the raw location data for the electrode locations as described, for example, in U.S. Pat. No. 7,263,397, which is hereby incorporated herein by reference in its entirety. The electrode data may also be used to compensate for changes in the impedance of the body of the patient as described, for example, in U.S. Pat. No. 7,885,707, which is also incorporated herein by reference in its entirety.

Therefore, in one representative embodiment, system 8 first selects a set of surface electrodes and then drives them with current pulses. While the current pulses are being delivered, electrical activity, such as the voltages measured with at least one of the remaining surface electrodes and in vivo electrodes, is measured and stored. Compensation for artifacts, such as respiration and/or impedance shifting, may be performed as indicated above.

In some embodiments, system 8 is the EnSite™ Velocity™ or EnSite Precision™ cardiac mapping and visualization system of Abbott Laboratories. Other localization systems, however, may be used in connection with the present teachings, including for example the RHYTHMIA HDX™ mapping system of Boston Scientific Corporation (Marlborough, Mass.), the CARTO navigation and location system of Biosense Webster, Inc. (Irvine, Calif.), the AURORA® system of Northern Digital Inc. (Waterloo, Ontario), Sterotaxis' NIOBE® Magnetic Navigation System (Stereotaxis, Inc., St. Louis, Mo.), as well as MediGuide™ Technology from Abbott Laboratories.

The localization and mapping systems described in the following patents (all of which are hereby incorporated by reference in their entireties) can also be used with the present invention: U.S. Pat. Nos. 6,990,370; 6,978,168; 6,947,785; 6,939,309; 6,728,562; 6,640,119; 5,983,126; and 5,697,377.

Aspects of the disclosure relate to graphically representing multiple electrophysiological characteristics on a single surface model. Accordingly, system 8 can also include a modeling module 58. Modeling module 58 can be used, inter alia, to graphically represent two or more electrophysiological characteristics (e.g., two or more electrophysiology maps) on a single geometric model (e.g., a single cardiac geometry).

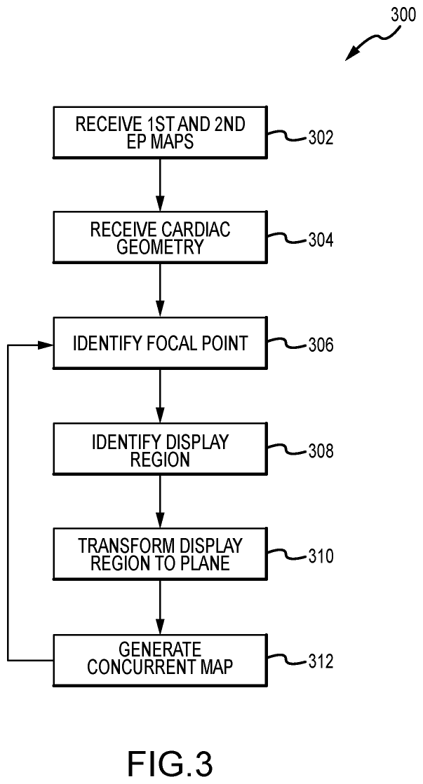

One exemplary method according to the present teachings will be explained with reference to the flowchart 300 of representative steps presented as FIG. 3. In some embodiments, for example, flowchart 300 may represent several exemplary steps that can be carried out by electroanatomical mapping system 8 of FIG. 1 (e.g., by processor 28 and/or modeling module 58). It should be understood that the representative steps described below can be either hardware- or software-implemented. For the sake of explanation, the term “signal processor” is used herein to describe both hardware- and software-based implementations of the teachings herein.

In block 302, system 8 receives two electrophysiology maps, a first electrophysiology map representing a first electrophysiological characteristic (e.g., local activation time) and a second electrophysiology map representing a second electrophysiological characteristic (e.g., peak-to-peak voltage). As discussed above, such maps will be familiar to the ordinarily skilled artisan, such that their acquisition, content, and the like need not be further explained herein.

Similarly, in block 304, system 8 receives a three-dimensional cardiac surface model (e.g., a cardiac geometry). Again, those of ordinary skill in the art will be familiar with such models, such that a detailed description thereof is not necessary herein.

Furthermore, those of ordinary skill in the art will also appreciate how to graphically represent either the first or second electrophysiology map individually on the surface model received in block 304. Aspects of the instant disclosure, however, relate to representing the first and second electrophysiology maps concurrently on the surface model received in block 304.

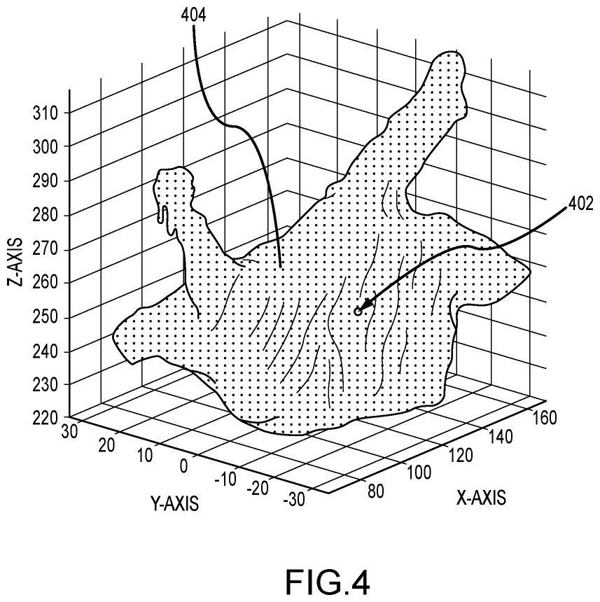

A focal point is identified in block 306. Various approaches to identifying the focal point are contemplated. For instance, in some aspects of the disclosure, a practitioner defines the focal point, such as via a point-and-click user interface on a graphical representation of the surface model received in block 304. In other aspects of the disclosure, system 8 defines the focal point based upon a viewing orientation of a graphical representation of the surface model, such that the focal point can change if and when the practitioner modifies the viewing orientation thereof. In still other aspects of the disclosure, the practitioner can specify a canonical representation (e.g., Left Anterior Oblique, Right Anterior Oblique, and so forth) that defines the viewing orientation, and thereby defines the focal point. For purposes of illustrating block 306, FIG. 4 depicts an exemplary focal point 402 on a three-dimensional cardiac surface model 404.

In block 308, a region around the focal point, referred to herein as the “display region,” is identified. Those skilled in the art will appreciate that, in order to transform a surface region from three dimensions into a plane, that region should have the topology of a disk. The display region, therefore, should have the topology of a disk.

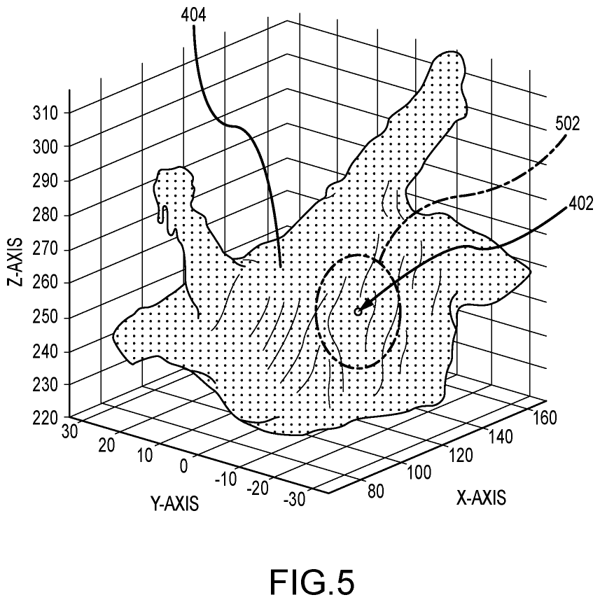

In embodiments of the disclosure, the display region is identified by propagating a geodesic wave front outward from the focal point along the surface model, adding to the display region any polygons (e.g., triangles) of the surface model through which the geodesic wavefront passes as it propagates. It is contemplated that the geodesic wavefront will be allowed to propagate until either (1) it reaches a preset geodesic distance (e.g., between about 4 cm and about 6 cm, and, in embodiments of the disclosure, about 5 cm) from the focal point or (2) it intersects itself. It should also be understood that the display region can contain holes or gaps, e.g., about anatomical features, such as the pulmonary veins. For purposes of illustrating block 308, FIG. 5 depicts an exemplary display region 502 around focal point 402.

In block 310, the display region is transformed from the three-dimensional surface model into a planar surface, and, in embodiments of the disclosure, into a planar surface that is tangent to the three-dimensional surface model at the focal point. More particularly, a continuous one-to-one mapping can be computed from the three-dimensional model to a plane using a transformation algorithm.

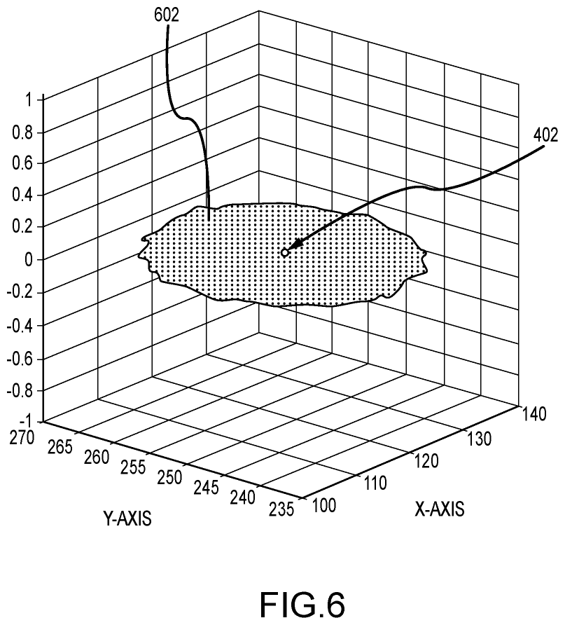

Such a transformation can introduce distortion (e.g., in area, distance, and/or angle), and it is desirable to utilize a transformation algorithm that trades off angle distortion in order to reduce distortion in area and distance in connection with the present teachings. In this regard, suitable transformation algorithms are disclosed in Michael S. Floater and Kai Hormann, “Surface Parameterization: A Tutorial and Survey” (2005); Bruno Levy et al., “Least Squares Conformal Maps for Automatic Texture Atlas Generation” (2002); and Ligang Liu et al., “A Local/Global Approach to Mesh Parameterization” (2008). These references, which will be familiar to those of ordinary skill in the art, are incorporated by reference as though fully set forth herein. For purposes of illustrating block 310, FIG. 6 depicts an exemplary plane 602, resulting from the application of a transformation algorithm to display region 502.

A concurrent map can be generated in block 312. As used herein, the term “concurrent map” refers to a graphical representation of at least two electrophysiological characteristics on a single surface model. Thus, for example, the concurrent map of block 312 can include a graphical representation of both the first electrophysiological map and the second electrophysiological map received in block 302.

The concurrent map can be represented graphically on the planar transform of the display region (e.g., on plane 602). More specifically, the elevation of the plane can be varied (e.g., raised or lowered in a direction normal to the plane) according to the values of one of the electrophysiological characteristics, while the second electrophysiological characteristic can be represented through the use of a color scale, grey scale, stippling, or the like.

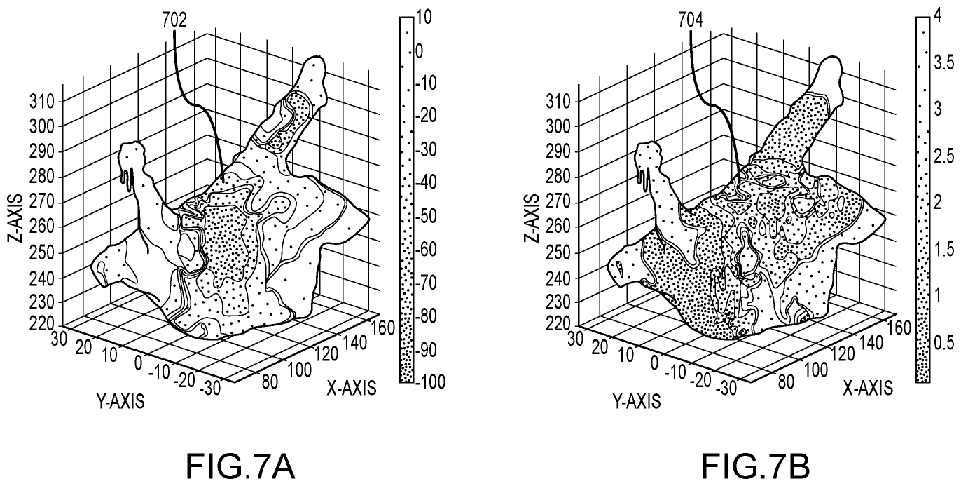

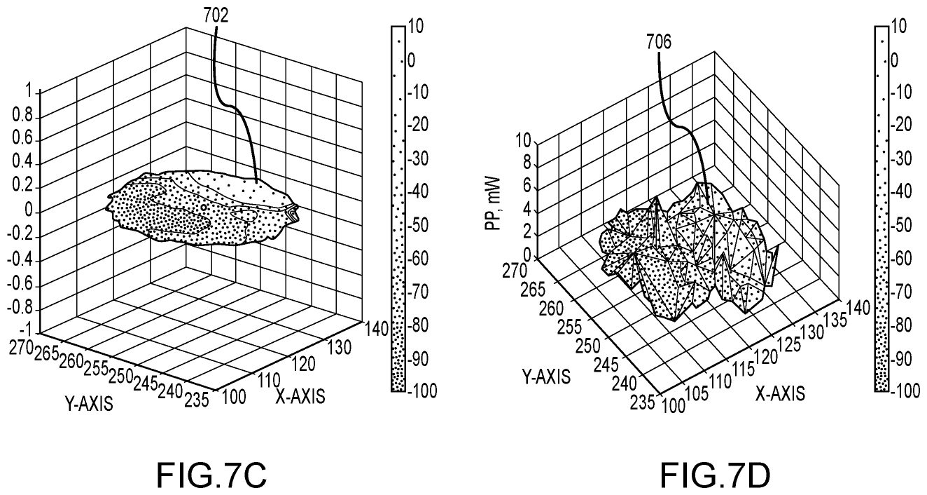

FIGS. 7A through 7D depict block 312. In particular, FIGS. 7A and 7B respectively depict a graphical representation of a local activation time map 702 and a peak-to-peak voltage map 704, both in greyscale. FIG. 7C depicts local activation time map 702 on plane 602, again in greyscale. FIG. 7D depicts both local activation time map 702, in greyscale, as well as peak-to-peak voltage map 704, expressed through variations in the elevation of plane 602. FIG. 7D, therefore, is a concurrent map 706 according to the instant teachings.

FIG. 3 also shows a loop back to block 306, indicating that the concurrent map can be updated whenever the focal point changes. As discussed elsewhere in this disclosure, a practitioner may change the focal point, such as by defining a new focal point through a point-and-click interface or by effectively redefining the focal point by changing the viewing orientation of the surface model or concurrent map.

The scale for the graphical representations of both electrophysiology maps in the concurrent map (e.g., the color scale or grey scale range, on the one hand, and the height range, on the other hand) can be automatically determined by system 8 according to the maximum and minimum values of the respective electrophysiological characteristics within the display area. Alternatively, the user can define either or both scales.

Although several embodiments have been described above with a certain degree of particularity, those skilled in the art could make numerous alterations to the disclosed embodiments without departing from the spirit or scope of this invention.

For example, the teachings herein can be applied in real time (e.g., during an electrophysiology study/as electrophysiology data points are collected) or during post-processing (e.g., to electrophysiology data points collected during an electrophysiology study performed at an earlier time). In either case, the concurrent map can be dynamically updated during a given beat, which will give the practitioner a visual sense of the propagation of a cardiac activation wavefront.

As another example, although the foregoing describes the identification of the focal point using a graphical representation of the three-dimensional model of the cardiac surface (e.g., via a point-and-click interface or based upon a viewing orientation of the model itself), the focal point can be identified using any graphical representation. For instance, the focal point can be identified (e.g., defined or redefined) via a point-and-click interface or based upon a viewing orientation of a graphical representation of the first electrophysiology map, of the second electrophysiology map, or of a concurrent map created according to the foregoing teachings.

As yet another example, although the foregoing exemplary embodiment of a concurrent map is described with reference to two electrophysiological characteristics, concurrent maps according to the instant teachings can be used to display any number of electrophysiological characteristics. In particular, it is contemplated that multiple electrophysiological characteristics can be represented through elevation changes, and that multiple electrophysiological characteristics can be represented on the elevation-varied plane through the use of different display conventions (including, in aspects of the disclosure, the use of glyphs).

As a still further example, the teachings herein can be applied to multiple focal points, which can be distributed in space (e.g., at different points on the surface model) and/or time (e.g., at different points in the cardiac cycle).

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A method of graphically representing multiple electrophysiological characteristics on a single surface model, the method comprising:
receiving a three-dimensional model of a cardiac surface;
identifying a focal point within the three-dimensional model of the cardiac surface;
identifying a display region of the three-dimensional model of the cardiac surface around the focal point;
transforming the display region from a three-dimensional model into a plane; and
graphically representing a first electrophysiological characteristic by varying an elevation of the plane according to values of the first electrophysiological characteristic.

2. The method according to claim 1, further comprising graphically representing a second electrophysiological characteristic on the elevation-varied plane.

3. The method according to claim 1, wherein identifying a display region of the three-dimensional model of the cardiac surface around the focal point comprises:
propagating a geodesic wavefront through the three-dimensional model of the cardiac surface, originating at the focal point; and
adding polygons of the three-dimensional model of the cardiac surface through which the geodesic wavefront passes to the display region.

4. The method according to claim 3, wherein propagating the geodesic wavefront through the three-dimensional model of the cardiac surface ends after the geodesic wavefront propagates a preset geodesic distance from the focal point.

5. The method according to claim 4, wherein the preset geodesic distance is between 4 cm and 6 cm.

6. The method according to claim 3, wherein propagating the geodesic wavefront through the three-dimensional model of the cardiac surface ends when the geodesic wavefront intersects itself.

7. The method according to claim 1, wherein transforming the display region from a three-dimensional model into a plane comprises computing a continuous one-to-one mapping from the three-dimensional model of the cardiac surface to a plane using a transformation algorithm.

8. The method according to claim 7, wherein the transformation algorithm comprises one of: least squares conformal mapping algorithm and a local/global approach to mesh parameterization algorithm.

9. The method according to claim 1, wherein identifying a focal point within the three-dimensional model of the cardiac surface comprises accepting user input designating the focal point within the three-dimensional model of the cardiac surface.

10. The method according to claim 1, wherein identifying a focal point within the three-dimensional model of the cardiac surface comprises identifying the focal point within the three-dimensional model of the cardiac surface according to a viewing orientation of the three-dimensional model of the cardiac surface.

11. The method according to claim 1, wherein the plane is tangent to the cardiac surface at the focal point.

12. The method according to claim 1, wherein varying an elevation of the plane according to values of the first electrophysiological characteristic comprises displacing points in the plane in a direction normal to the plane according to values of the first electrophysiological characteristic.

13. A method of graphically representing two electrophysiology maps in a single representation, the method comprising:
receiving a first electrophysiology map of a first electrophysiological characteristic;
receiving a three-dimensional cardiac surface model;
identifying a focal point in the three-dimensional cardiac surface model;
transforming a display region about the focal point from a three-dimensional cardiac surface into a plane; and
graphically representing the first electrophysiology map of the first electrophysiological characteristic by varying an elevation of the plane.

14. The method according to claim 13, further comprising:
receiving a second electrophysiology map of a second electrophysiological characteristic; and
graphically representing the second electrophysiology map of the second electrophysiological characteristic on the elevation-varied plane.

15. The method according to claim 13, wherein transforming a display region about the focal point from a three-dimensional cardiac surface into a plane comprises:
identifying the display region by propagating a geodesic wavefront from the focal point; and
computing a continuous one-to-one mapping from the three-dimensional model of the cardiac surface to a plane using a transformation algorithm.

16. The method according to claim 15, wherein propagating a geodesic wavefront from the focal point comprises propagating the geodesic wavefront from the focal point to a preset geodesic distance from the focal point.

17. The method according to claim 15, wherein propagating a geodesic wavefront from the focal point comprises propagating the geodesic wavefront from the focal point until the geodesic wavefront intersects itself.

18. The method according to claim 15, wherein identifying the display region by propagating a geodesic wavefront from the focal point comprises adding polygons of the three-dimensional model of the cardiac surface passed by the propagating geodesic wavefront to the display region.

19. A system for graphically representing multiple electrophysiological characteristics on a single surface model, the system comprising:
a modeling module configured to:
receive a three-dimensional model of a cardiac surface, the three-dimensional model including a focal point;
identify a display region of the three-dimensional model of the cardiac surface around the focal point;
transform the display region from the three-dimensional model into a plane; and
output a graphical representation of a first electrophysiological characteristic by varying an elevation of the plane according to values of the first electrophysiological characteristic.

20. The system according to claim 19, wherein the modeling module is further configured to output a graphical representation of a second electrophysiological characteristic on the elevation-varied plane.
